(19) United States
(12) Patent Application Publication
(10) Pub. No.: US 2024/0050331 A1
(43) Pub. Date: Feb. 15, 2024

(54) PROCESS FOR PREPARING MICROCAPSULES

(71) Applicant: Firmenich SA, Satigny (CH)

(72) Inventors: Nuerxida PULATI, Plainsboro, NJ (US); Huda JERRI, Plainsboro, NJ (US); Nicholas IMPELLIZZERI, Plainsboro, NJ (US); Christopher HANSEN, Plainsboro, NJ (US)

(21) Appl. No.: 18/259,066

(22) PCT Filed: Dec. 14, 2021
(86) PCT No.: PCT/EP2021/085661

§ 371 Date: Jun. 22, 2023

Related U.S. Application Data
(60) Provisional application No. 63/130,162, filed on Dec. 23, 2020.

(30) Foreign Application Priority Data
Jan. 13, 2020 (EP) 21151391.6

(51) Int. Cl. A61K 8/11; A61Q 13/00; A61K 8/64; A61K 8/73; A61K 8/58; A61K 8/92; A61Q 5/12; A61Q 5/02; A61Q 15/00; A61Q 19/10; B01J 13/14; C11D 17/00; C11D 3/50; C11D 3/00; C11D 11/00

(52) U.S. Cl. CPC A61K 8/11; A61Q 13/00; A61K 8/64; A61K 8/736; A61K 8/58; A61K 8/92; A61Q 5/12; A61Q 5/02; A61Q 15/00; A61Q 19/10; B01J 13/14; C11D 17/0039; C11D 3/505; C11D 3/001; C11D 11/0017; A61K 2800/412; A61K 2800/56; A61K 2800/874; A61K 2800/65

(57) ABSTRACT

The present invention relates to a new process for the preparation of core-shell microcapsules. Microcapsules are also an object of the invention. Consumer products comprising said microcapsules, in particular perfumed consumer products or flavoured consumer products are also part of the invention.

### TECHNICAL FIELD

The present invention relates to a new process for the preparation of core-shell microcapsules. Microcapsules are also an object of the invention. Consumer products comprising said microcapsules, in particular perfumed consumer products or flavoured consumer products are also part of the invention.

### BACKGROUND OF THE INVENTION

One of the problems faced by the perfume and flavour industry lies in the relatively rapid loss of olfactive benefit provided by active compounds due to their volatility. The encapsulation of those active substances provides at the same time a protection of the ingredients there-encapsulated against “aggressions” such as oxidation or moisture and allows, on the other hand, a certain control of the kinetics of flavour or fragrance release to induce sensory effects through sequential release.

Polyurea and polyurethane-based microcapsule slurry are widely used for example in perfumery industry for instance as they provide a long lasting pleasant olfactory effect after their applications on different substrates. Those microcapsules have been widely disclosed in the prior art (see for example WO2007/004166 or EP 2300146 from the Applicant).

In addition to the performance in terms of stability and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention is proposing a solution to the above-mentioned problem, based on new core-shell microcapsules comprising a biopolymer membrane that provides a scaffold to attract silicon precursors. As a result, silicon precursors can silicify the biopolymer membrane and/or form a silicified membrane around the biopolymer membrane.

### BRIEF DESCRIPTION OF THE FIGURES

FIG. 1 represents scanning electron micrograph of Microcapsules A according to the invention.

FIG. 2 represent silicon (Si) elemental EDS (energy dispersive spectroscopy) mapping of Microcapsules A in FIG. 1 (FIG. 2a) and SEM-EDS spectra of the region (FIG. 2b).

FIG. 3 represents optical image of Microcapsules A, dried on a glass slide.

FIG. 4 represents scanning electron micrograph of Microcapsules B according to the invention.

FIG. 5 represents scanning electron micrograph of Microcapsules C according to the invention.

FIG. 6 represents scanning electron micrograph of Microcapsules D according to the invention.

FIG. 7 represents scanning electron micrograph of Microcapsules H according to the invention.

FIG. 8 represents scanning electron micrograph of Microcapsules J, imaged after rinsing and drying, according to the invention.

FIG. 9 represents scanning electron micrograph of Microcapsules J, after spray drying (coarse collection).

FIG. 10 represents scanning electron micrograph of spray dried Microcapsules J (coarse collection), after resuspension in water to show capsules are intact when carrier is dissolved.

FIG. 11 represents optical image of Microcapsules K, dried on a glass slide, according to the invention.

FIG. 12 represents scanning electron micrograph of Microcapsules K, according to the invention.

FIG. 13 represents optical image of Microcapsules L dried on a glass slide, according to the invention.

FIG. 14 represents scanning electron micrograph of Microcapsules L, according to the invention.

FIG. 15 represents ratios of headspace intensities for Microcapsules L, M, and O respectively, dosed onto paper blotters and evaluated before and after applying friction to demonstrate pop effect.

FIG. 16a and FIG. 16b represent scanning electron micrograph of Microcapsules A which are intact after incubation in fabric softener for two months at 37° C. in a closed jar.

FIG. 17 represent back scattering electron micrograph of Microcapsules A after heating at 500° C. (FIG. 17a), corresponding area silicon (Si) elemental EDS (energy dispersive spectroscopy) mapping (FIG. 17b) and SEM-EDS spectra (FIG. 17c) to illustrate presence of silicon in shells after extreme heat treatment.

### DETAILED DESCRIPTION OF THE INVENTION

Indeed, it has now been found that microcapsules encapsulating hydrophobic materials, preferably active ingredients, could be obtained by complexation of a protein with a polycation (for example between whey protein isolate with chitosan oligosaccharide) and using said complex as an emulsifier to create an oil-in-water emulsion. A biopolymer shell made of said complexes can be formed at the oil/water interface. Silicon precursors can be attracted to or within the biopolymer membrane and silicify the biopolymer membrane.

The process of the invention therefore provides a solution to the above-mentioned problems as it allows preparing microcapsules with the desired physical integrity and stability in different applications.

Unless stated otherwise, percentages (%) are meant to designate a percentage by weight of a composition.

By “hydrophobic material”, it is meant a material which forms a two-phase dispersion when mixed with water. According to the invention, the hydrophobic material can be “inert” material like solvents or active ingredients. According to an embodiment, the hydrophobic material is a hydrophobic active ingredient.

By “active ingredient”, it is meant a single compound or a combination of ingredients.

By “perfume oil or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles, aggregates, precipitates, complexes and/or emulsion droplets are dispersed in a continuous phase of a different composition and it specifically includes a suspension or an emulsion. “Dispersion” according to the invention can encompass two-phases dispersion or multiple dispersion (more than 2-phases).

A “core-shell microcapsule”, or the similar, in the present invention it is meant that capsules have a particle size distribution in the micron range (e.g. a mean diameter (d(v, 0.5)) preferably comprised between about 1 and 3000 microns) and comprise a shell and an internal continuous oil phase enclosed by the shell. According to the invention, the wordings “mean diameter” or “mean size” are used indifferently.

Microcapsules of the present invention have a mean size preferably greater than 10 microns, more preferably greater than 15 microns, even more preferably greater than 20 microns.

According to an embodiment, microcapsules have a mean size comprised between 10 and 500 microns, preferably between 10 and 100 microns, more preferably between 10 and 50 microns.

By “microcapsule slurry”, it is meant microcapsule(s) that is (are) dispersed in a liquid. According to an embodiment, the slurry is an aqueous slurry, i.e the microcapsule(s) is (are) dispersed in an aqueous phase.

By “biopolymer membrane” or “biopolymer shell”, it is meant a layer comprising a complex between a protein and a polycation, preferably an organic polycation.

By “polycation”, it is meant multivalent cation or molecule having more than one positive charge.

By “polyfunctional monomer”, it is meant a molecule that, as a unit, reacts or binds chemically to form a polymer or supramolecular polymer. The polyfunctional monomer defined in the present invention has at least two functions capable of forming a microcapsule shell.

By “protein”, it is meant a single protein or a combination of proteins.

By “whey protein isolate”, it is meant greater than 90% protein by weight, and processed to remove the fat and lactose.

By “protein/polycation complexes”, it is meant materials formed through the interaction between protein and polycation. Complexes, precipitates, particles or aggregates are used indifferently in the present invention.

By “chitosan oligosaccharide”, it is meant an oligomer of chitosan with an average molecular weight (MW) preferably below 5,000 Da.

By “composite shell”, it is meant that the shell is composed from two or more materials.

According to a particular embodiment, the microcapsule shell has an inner layer formed of biopolymer-based material and an outer layer formed of the silicon-based material. According to a particular embodiment, the microcapsule shell has an inner layer richer in biopolymer-based material and an outer layer richer in the silicon-based material. According to an embodiment, the inner and the outer layers are interlinked layers, it is meant a shell consisting of layers that are linked by chemical or physical interactions, thereby forming a composite structure. As physical or chemical interactions, one may cite covalent bonds, ionic bonds, coordinate covalent bonds, hydrogen bonds, van der Waals interaction, hydrophobic interactions, chelation, or steric effects.

Thermal processing, heat treatments and annealing may be further used to promote physical interactions to form the composite structure.

By “silicon-based material”, it is meant material containing elemental silicon.

### Process for Preparing a Core-Shell Microcapsule Slurry

In a first aspect, the present invention relates to a process for preparing a core-shell microcapsule slurry, said process comprising the steps of:
(i) Mixing in a dispersing phase a protein and a polycation;
(ii) Adding an oil phase comprising a hydrophobic material, preferably a perfume or a flavor to the dispersing phase to form a dispersion;
(iii) performing a curing step to form a microcapsule slurry;
wherein at least one silicon precursor is added in step (i) and/or in step (ii) and/or in step (iii).

According to an embodiment, the dispersion obtained in step ii) is a two-phases dispersion. Thus, according to a particular embodiment, the process comprises the steps of:
(i) Mixing in a dispersing phase a protein and a polycation;
(ii) Adding an oil phase comprising a hydrophobic material, preferably a perfume or a flavor to the dispersing phase to form a two-phases dispersion;
(iii) performing a curing step to form a microcapsule slurry;
wherein at least one silicon precursor is added in step (i) and/or in step (ii) and/or in step (iii).

Without being bound by any theory, the inventors are of the opinion that the protein (for example whey protein isolate (WPI) and the polycation (for example chitosan oligosaccharide) can undergo complexation due to the favorable interaction including electrostatic interaction between negatively charged moieties of the protein and positively charged moieties of the polycation.

The protein/polycation complex can act as an emulsifier and form a biopolymer membrane at the oil in water interface. Then, the formed biopolymer membrane can provide silicification scaffold/attract silicon precursor to form a composite shell comprising both a silicon-based material and a biopolymer-based material (made of complexes between protein and the polycation).

In a first step of the process, a protein and a polycation is mixed in a dispersing phase. It should be understood that the protein and the polycation have attractive interactions and form complexation. Natural materials have multiple different functional groups and moieties which can interact with other materials through electrostatics, hydrogen bonding, van der Waals interactions over a range of pH, ionic strength, temperature, solution and processing conditions.

The person skilled in the art will be able to select suitable conditions to form complexes.

Said protein and said polycation are mixed under conditions sufficient to form a suspension of complexes between the protein and the polycation. Typically, the mixing step is carried out at a pH between 4 and 8, preferably between 4 and 7, most preferably between 5 and 6.

According to the invention, the protein and the polycation are able to interact to form complex. Typically, the protein is negatively charged and the polycation is positively charged.

According to a particular embodiment, the protein is chosen in the group consisting of whey protein, (preferably whey protein isolate), milk proteins, caseinate salts such as sodium caseinate or calcium caseinate, casein, hydrolyzed proteins, gelatins, gluten, pea protein, soy protein, silk protein, beta-lactoglobulin, ovalbumine, bovine serum albumin, and mixtures thereof.

According to an embodiment, the protein is chosen in the group consisting of potato protein, chickpea protein, algae protein, faba bean protein, barley protein, oat protein, wheat gluten protein, lupin protein, and mixtures thereof.

According to an embodiment, the protein is chosen in the group consisting of potato protein, chickpea protein, algae protein, faba bean protein, barley protein, oat protein, wheat gluten protein, lupin protein, whey protein (preferably whey protein isolate), milk proteins, caseinate salts such as sodium caseinate or calcium caseinate, casein, hydrolyzed proteins, gelatins, gluten, pea protein, soy protein, silk protein, beta-lactoglobulin, ovalbumine, bovine serum albumin and mixtures thereof.

According to a particular embodiment, the polycation is chosen in the group consisting of chitosan, chitosan oligomer, chitosan oligosaccharide, a cation such as Ca²⁺, Mg²⁺, Zn²⁺, Ba²⁺, Sr²⁺, and mixtures thereof.

When a cation is added as polycation (for example as Ca²⁺, Mg²⁺, Zn²⁺, Ba²⁺, Sr²⁺, it should be understood that it is added in the form of a salt such as CaCl₂, CaBr₂, CaI₂, calcium acetate, calcium lactate, Ca(NO₃)₂, Mg(NO₃)₂, MgCl₂, MgBr₂, MgI₂, magnesium acetate, ZnCl₂, ZnBr₂, ZnI₂, Zn(NO₃)₂, ZnSO₄, zinc acetate, BaCl₂, Sr(NO₃)₂, SrCl₂, SrBr₂, SrI₂ and strontium acetate.

According to a particular embodiment, the polycation is chitosan oligosaccharide.

The chitosan oligosaccharide has preferably a low molecular weight, preferably below 5000, preferably below 3000.

According to a particular embodiment, the protein is whey protein isolate and the polycation is chitosan oligosaccharide.

According to a particular embodiment, a cation, preferably chosen in the group consisting of Ca²⁺, Mg²⁺, Zn²⁺, Ba²⁺, Sr²⁺ is added (in the form of a salt) in addition of the protein.

According to a particular embodiment, the dispersing phase comprises a protein, a cation (added in the form of a salt) and chitosan oligosaccharide.

According to a particular embodiment, the weight ratio in the slurry between the protein and the polycation is comprised between 5:1 and 1:3, preferably between 3:1 and 1:2, more preferably is 2:1.

There is no restriction regarding the nature of the solvent that can be used in step i) as long as it can dissolve/disperse the protein/polycation complexes.

According to a particular embodiment, the dispersing phase comprises, preferably consists of water.

According to another particular embodiment, the content of water is below or equal to 10%, preferably below or equal to 5%, more preferably below or equal to 3% by weight based on the total weight of the dispersing phase.

According to a particular embodiment, the dispersing phase is free of water.

According to an embodiment, the dispersing phase comprises a solvent chosen in the group consisting of glycerol, 1,4-butanediol, ethylene glycol and mixtures thereof.

In a second step, an oil phase comprising a hydrophobic material, preferably a perfume or a flavor is added to the dispersing phase to form a dispersion, preferably a two-phases dispersion, wherein the mean droplet size is preferably comprised between 1 and 1000 microns, more preferably between 1 and 500 microns, and even more preferably between 5 and 50 microns.

The protein/polycation complexes formed in step i) act as an emulsifier to form the two-phases dispersion. Any known techniques such as homogenization, sonication, shearing mixing and stirring can be used to form the dispersion.

### Hydrophobic Material

The hydrophobic material according to the invention can be “inert” material like solvents or active ingredients. A single hydrophobic material or mixture of hydrophobic materials can be used.

When the hydrophobic material is an active ingredient, it is preferably chosen from the group consisting of flavors, flavor ingredients, perfumes, perfume ingredients, nutraceuticals, cosmetics, pest control agents, malodor counteracting ingredient, biocide actives and mixtures thereof. The term “malodor counteracting ingredient” is understood as being capable of reducing the perception of malodor, i.e. of an odor that is unpleasant or offensive to the human nose.

According to a particular embodiment, the hydrophobic material comprises a phase change material (PCM).

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, modulators, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils (for example Thyme oil), and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2,7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 3-(3,3/1,1-dimethyl-5-indanyl)propanal, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0²,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl)butanal.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, triethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance (bulky materials) and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a log P value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3RS,3aRS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), (1′R)-2-[2-(4′-methyl-3′-cyclohexen-1′-yl)propyl]cyclopentanone, alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0^{2,6}]dec-3-en-8-yl 2-methylpropanoate and (1RS,2SR,6RS,7RS,8SR)-tricyclo[5.2.1.0^{2,6}]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0²,7]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), 9-hexadecen-16-olide (origin: Firmenich SA, Geneva, Switzerland), pentadecenolide (origin: Firmenich SA, Geneva, Switzerland), 3-methyl-(4/5)-cyclopentadecenone, origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

According to a particular embodiment, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<−4, and
0-75 wt % of a density balancing material having a density greater than 1.07 g/cm³. “High impact perfume raw materials” should be understood as perfume raw materials having a Log T<−4. The odor threshold concentration of a chemical compound is determined in part by its shape, polarity, partial charges and molecular mass. For convenience, the odor threshold concentration is presented as the common logarithm of the threshold concentration, i.e., Log [Threshold] (“Log T”).

A “density balancing material” should be understood as a material having a density greater than 1.07 g/cm³ and having preferably low or no odor.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.

The nature of high impact perfume raw materials having a Log T<−4 and density balancing material having a density greater than 1.07 g/cm³ are described in WO2018115250, the content of which is included by reference.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl)ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3(4H)-one, (2E,6Z)-2,6-nonadien-1-ol, (4Z)-4-dodecenal, (+−)-4-hydroxy-2,5-dimethyl-3(2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl)phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one(2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2(3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl)ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl)pent-1-en-3-one, indol, 7-propyl-2H,4H-1,5-benzodioxepin-3-one, ethyl praline, (4-methylphenoxy)acetaldehyde, ethyl tricyclo[5.2.1.0.^{2,6}]decane-2-carboxylate, (+)-(l'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxides, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4.

According to an embodiment, perfume raw materials having a Log T<−4 comprise between and 70% by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-1(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

According to an embodiment, the perfume formulation comprises
0 to 60 wt. % of a hydrophobic solvent (based on the total weight of the perfume formulation),
to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of bulky materials of groups 1 to 6, preferably 3 to 6 as previously defined and
   at least 15%, preferably at least 20%, more preferably at least 25%, even more preferably at least 30% of high impact perfume materials having a Log T<−4, optionally, further hydrophobic active ingredients.
      According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜ-δD_{fragrance})²⁺(δPₛₒₗᵥₑₙₜ-δP_{fragrance})²⁺(δHₛₒₗᵥₑₙₜ-δH_{fragrance})²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

In a particular embodiment, at least 90% of the perfume oil, preferably at least 95% of the perfume oil, most preferably at least of 98% of the perfume oil has at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

According to an embodiment, the perfuming formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a clogP of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably, as examples the following ingredients can be listed as modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-1(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

By “flavor oil”, it is meant here a flavoring ingredient or a mixture of flavoring ingredients, solvents or adjuvants of current use for the preparation of a flavoring formulation, i.e. a particular mixture of ingredients which is intended to be added to an edible composition or chewable product to impart, improve or modify its organoleptic properties, in particular its flavor and/or taste. Flavoring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavoring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, van Nostrand Co., Inc. Solvents and adjuvants of current use for the preparation of a flavoring formulation are also well known in the art.

In a particular embodiment, the flavor is a mint flavor. In a more particular embodiment, the mint is selected from the group consisting of peppermint and spearmint.

In a further embodiment, the flavor is a cooling agent or mixtures thereof.

In another embodiment, the flavor is a menthol flavor.

Flavors that are derived from or based on fruits where citric acid is the predominant, naturally-occurring acid include but are not limited to, for example, citrus fruits (e.g. lemon, lime), limonene, strawberry, orange, and pineapple. In one embodiment, the flavors food is lemon, lime or orange juice extracted directly from the fruit. Further embodiments of the flavor comprise the juice or liquid extracted from oranges, lemons, grapefruits, key limes, citrons, clementines, mandarins, tangerines, and any other citrus fruit, or variation or hybrid thereof. In a particular embodiment, the flavor comprises a liquid extracted or distilled from oranges, lemons, grapefruits, key limes, citrons, clementines, mandarins, tangerines, any other citrus fruit or variation or hybrid thereof, pomegranates, kiwifruits, watermelons, apples, bananas, blueberries, melons, ginger, bell peppers, cucumbers, passion fruits, mangos, pears, tomatoes, and strawberries.

In a particular embodiment, the flavor comprises a composition that comprises limonene, in a particular embodiment, the composition is a citrus that further comprises limonene.

In another particular embodiment, the flavor comprises a flavor selected from the group comprising strawberry, orange, lime, tropical, berry mix, and pineapple.

The phrase flavor includes not only flavors that impart or modify the smell of foods but include taste imparting or modifying ingredients. The latter do not necessarily have a taste or smell themselves but are capable of modifying the taste that other ingredients provides, for instance, salt enhancing ingredients, sweetness enhancing ingredients, umami enhancing ingredients, bitterness blocking ingredients and so on.

In a further embodiment, suitable sweetening components may be included in the particles described herein. In a particular embodiment, a sweetening component is selected from the group consisting of sugar (e.g., but not limited to sucrose), a stevia component (such as but not limited to stevioside or rebaudioside A), sodium cyclamate, aspartame, sucralose, sodium saccharine, and Acesulfam K or mixtures thereof.

According to any one of the invention's embodiments, the hydrophobic material represents between about 10% and 60% w/w, or even between 15% and 45% w/w, by weight, relative to the total weight of the dispersion as obtained after step ii).

According to a particular embodiment, shellac is added to the oil phase.

According to the invention, at least one silicon precursor is added in step (i) and/or in step (ii) and/or in step (iii).

The silicon precursor may be chosen in the group consisting of tetraethyl orthosilicate (TEOS), tetramethyl orthosilicate (TMOS), triethoxymethylsilane, dimethyldimethoxysilane, ethyltriethoxysilane, amine functionalized silanes, (3-aminopropyl)triethoxysilane, (3-Aminopropyl)trimethoxysilane, N-(2-aminoethyl)-3-aminopropyltriethoxysilane, N-dimethyl-3-aminopropylmethyldimethoxysilane, 3-aminopropylmethyldiethoxysilane, 4-aminobutylthiethoxysilane, and mixtures thereof.

According to a particular embodiment, the at least one silicon precursor, preferably tetraethyl orthosilicate, is added in the dispersion, preferably in the two-phases dispersion obtained in step (ii).

According to a particular embodiment, the silicon precursor is pre-hydrolyzed (typically by mixing the silicon precursor with 1-5 mM HCl) so as to readily interact with biopolymer membrane.

According to a particular embodiment, the silicon precursor is added directly in the oil phase.

According to a particular embodiment, at least a first silicon precursor S1 is added in the dispersion, preferably in the two-phases dispersion obtained in step (ii), and at least a second silicon precursor S2 is added in step (iii).

According to a particular embodiment, at least a first silicon precursor S1 is added in the dispersion, preferably in the two-phases dispersion obtained in step (ii), and at least a second silicon precursor S2 is added during or after step (iii).

According to a particular embodiment, the first silicon precursor S1 is tetraethyl orthosilicate and the second silicon precursor S2 is (3-aminopropyl)triethoxysilane.

According to an embodiment, when present, the first silicon precursor S1 is added in an amount comprised between greater than 0% and 20%, preferably between 5% and 15% by weight based on the total weight of the dispersion.

According to an embodiment, when present, the second silicon precursor S2 is added in an amount comprised between greater than 0% and 10%, preferably between 1% and 6% by weight based on the total weight of the dispersion.

According to an embodiment, long and/or medium chain silane or mixture of silanes are added to the oil phase. Long and/or medium chain silane or mixture of silanes can be defined as silanes having and organic chain substitution of more than 3 carbons. Silanes with organic chain substitution of more than 3 carbons can be chosen from the group of silanes with organic chain substitution of more than 3 carbons such as triethoxy-n-octylsilane, dodecyltriethoxysilane, octadecyltriethoxysilane, decyltriethoxysilane, n-hexyltriethoxysilane and hexadecyltriethoxysilane, and mixtures thereof.

According to a particular embodiment, the oil phase is free from any polyfunctional monomer, preferably chosen in the group consisting of at least one polyisocyanate, poly anhydride (such as poly maleic anhydride), poly acid chloride (i.e acyl chloride), polyepoxide, acrylate monomers, and mixtures thereof.

According to another embodiment, a polyfunctional monomer, preferably chosen in the group consisting of at least one polyisocyanate, poly anhydride (such as poly maleic anhydride), poly acid chloride (i.e acyl chloride), polyepoxide, acrylate monomers, mixtures thereof is added in the oil phase.

According to a particular embodiment, the monomer added in step i) is at least one polyisocyanate having at least two isocyanate functional groups.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the monomer used in the process of the invention is present in amounts representing from 0.1 and 15%, preferably from 0.5 and 3% by weight based on the total amount of the oil phase.

In another step of the process, a curing step iii) is carried out which allows ending up with microcapsules in the form of a slurry.

According to an embodiment, in step iii) of the process, a heating step is carried out.

The purpose of this heating step is to denature the protein and induce the aggregation of the protein/polycation complexes at the oil-water interface, and also thermally anneal the shell.

The heating step can be carried out at a temperature T_{d}en (denaturation temperature of the protein), preferably comprised between 50° C. and 100° C., more preferably between 70° C. and 90° C. The duration of the heating step will depend on the heating temperature. Typically, the duration of the heating step is comprised between 60 and 180 minutes.

According to the nature of the protein, the person skilled in the art will be able to find a suitable temperature to induce the denaturation of said protein.

As non-limiting examples, the denaturation temperature T_{den} of:
whey protein is comprised between 70 and 90° C.
soy protein is comprised between 70 and 90° C.
bovine serum albumin is comprised between 50 and 82° C.
ovalbumin is comprised between 68 and 80° C.
potato protein is comprised between 50 and 90° C.
   This heating step can also enhance silicon precursor further hydrolysis and interaction with the biopolymeric membrane.
   It should be understood that during the curing step, silicon precursors penetrate to the biopolymer membrane, silicify the membrane, and further condense silicon on the shell. As a result, the shell formed is likely a composite biopolymer silicon shell, with the gradient of difference organic and silicon throughout the shell.

The heating step is preferably performed at a pH comprised between 4 and 6, more preferably between 4.5 and 5.5.

According to a particular embodiment, a cross-linker is added in at least one of the step of the process. According to a particular embodiment, the cross-linker can be added during the curing step iii) and/or after the curing step iii).

The cross-linker can be an enzymatic cross-linker such as an enzyme or a non-enzymatic cross-linker such as glutaraldehyde or genipin.

According to a particular embodiment, the cross-linker is an enzyme.

According to a particular embodiment, the enzyme is transglutaminase.

The enzyme may be used in an amount comprised between 0.001 and 5%, preferably between 0.001 to 1%, preferably between 0.001 and 0.1%, preferably between 0.005 and 0.02% based on the total weight of the slurry of step c).

According to a particular embodiment of the invention, at the end of step iii) or during step iii) one may also add to the invention's slurry a polymer selected from the group consisting of a non-ionic polysaccharide, a cationic polymer, a polysuccinimide derivative (as described for instance in WO2021185724) and mixtures thereof to form an outer coating to the microcapsule.

Non-ionic polysaccharide polymers are well known to a person skilled in the art and are described for instance in WO2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar@ (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step iii). It is clearly understood by a person skilled in the art that only part of said added polymers will be incorporated into/deposited on the microcapsule shell.

### Multiple Microcapsules System

According to an embodiment, the microcapsules of the invention (first microcapsule slurry) can be used in combination with a second microcapsules slurry.

Another object of the invention is a microcapsule delivery system comprising:
the microcapsule slurry of the present invention as a first microcapsule slurry, and
a second microcapsule slurry, wherein the microcapsules contained in the first microcapsule slurry and the second microcapsule slurry differ in their hydrophobic material and/or their wall material and/or in their coating material.

### Process for Preparing a Microcapsule Powder

Another object of the invention is a process for preparing a microcapsule powder comprising the steps as defined above and an additional step consisting of submitting the microcapsule slurry obtained in step iii) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, gum Arabic, vegetable gums, pectins, xanthans, alginates, carrageenans or cellulose derivatives to provide microcapsules in a powder form.

According to a particular embodiment, the carrier material contains free perfume oil which can be the same or different from the perfume from the core of the microcapsules.

### Microcapsule Slurry/Microcapsule Powder

Microcapsule slurry and microcapsule powder obtainable by the processes above-described are also an object of the invention.

In another aspect, the invention relates to a core-shell microcapsule comprising:
an oil-based core comprising a hydrophobic material, and
a composite shell comprising
   (i) a biopolymer-based material comprising complexes made of a protein and a polycation, and
   (ii) a silicon-based material According to an embodiment, the microcapsule has a positive zeta potential greater than 10 mV, preferably between 10 and 80, more preferably between 10 and 65 mV.

All the previous embodiments described previously for the process for preparing the microcapsule slurry also apply for the microcapsule slurry described above.

The definitions of hydrophobic material, protein, the polycation are the same as described hereinabove.

According to an embodiment, the silicon-based material is derived from a compound chosen from the group consisting of tetraethyl orthosilicate (TEOS), tetramethyl orthosilicate (TMOS), Triethoxymethylsilane, dimethyldimethoxysilane, ethyltriethoxysilane, amine functionalized silanes, (3-aminopropyl)triethoxysilane, (3-aminopropyl)trimethoxysilane, N-(2-aminoethyl)-3-aminopropyltriethoxysilane, N-Dimethyl-3-aminopropylmethyldimethoxysilane, 3-aminopropylmethyldiethoxysilane, 4-Aminobutylthiethoxysilane, and mixtures thereof.

According to the invention, the oil-based core comprises a hydrophobic material as defined previously.

In a particular embodiment, the shell material comprises a biodegradable material.

In a particular embodiment, the shell has a biodegradability of at least 40%, preferably at least 45%, 50%, 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and optionally coating may have a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

### Perfuming Composition/Consumer Products

The microcapsules of the invention can be used in combination with active ingredients. An object of the invention is therefore a composition comprising:
(i) microcapsules as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The microcapsules of the invention can be used for the preparation of perfuming or flavouring compositions which are also an object of the invention.

Another object of the present invention is a perfuming composition comprising:
(i) microcapsules as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.

As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) microcapsule slurry as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer product can be a perfume, such as a fine perfume, a cologne, an after-shave lotion, a body-splash; a fabric care product, such as a liquid or solid detergent, tablets and unit dose (single chamber or multi chambers), a fabric softener, a dryer sheet, a fabric refresher, an ironing water, or a bleach; a personal-care product, such as a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules as defined above or the perfuming composition as defined above,
wherein the consumer product is in the form of a personal care composition.

Personal care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), a skin-care product (e.g. a perfumed soap (for example a soap bar), shower or bath mousse, body wash, oil or gel, bath salts, or a hygiene product); or an oral care product such as tooth paste, tooth powders, oral whitening.

Another object of the invention is a consumer product comprising:
a home care or a fabric care active base, and
microcapsules as defined above or the perfuming composition as defined above,
wherein the consumer product is in the form of a home care or a fabric care composition.

Home care or fabric care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course, the above concentrations may be adapted according to the benefit effect desired in each product.

According to a particular embodiment, the consumer product in which the microcapsules are incorporated has a pH lower than 4.5.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkaline agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules, preferably in the form of a slurry, as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Example 1

### Preparation of Microcapsules by the Process of the Invention

### Material and Methods

### Electron Microscopy of Capsules:

Diluted microcapsules slurries were dried on carbon tape, which was adhered to aluminum stubs and sputter coated with gold palladium plasma. The stubs were placed into a scanning electron microscope (JOEL 6010 PLUS LA) for analysis. Energy dispersive spectroscopy (EDS) is used to identify the elemental composition of the sample area through point analyses, mapping and generation of spectra for regions of interest.

Optical microscopy of capsules:

Diluted microcapsules slurries were dried on glass slides and imaged using an optical microscope (Olympus EX51) with 10× and 20× objectives.

### General Protocol

1) Whey protein isolate and/or chitosan oligosaccharide are added to DI water at RT and stirred for about 30 mins at RT, until no dry powder or dry materials observed in the solution.
2) Optionally, CaCl₂).2H₂O is dissolved in DI water, and slowly added to whey protein isolate solution while stirring;
3) The perfume oil is combined with WPI/Chitosan Oligosaccharide (or CaCl₂).2H₂O) solution, and homogenized (10,000 rpm for 2 mins) to create oil-in-water emulsion.
4) The emulsion is transferred to a reactor and stirred at RT;
5) Meanwhile, TEOS is added to 1 mM HCl solution in a separate vial and sonicated for 20 mins;
6) The pre-hydrolyzed TEOS solution is added to the reactor; and the emulsion is stirred at RT for 30 mins;
7) Then the reactor is heated up to 80° C. and held at 80° C. for 2 hours before cooling to RT;
8) Optionally, APTES is added to the reactor, and the emulsion is stirred at RT for 20 hrs.

Microcapsules A-D were prepared according to the protocol described above and using the following components.

TABLE 1
Microcapsules composition

| | Micro- | Micro- | Micro- | Micro- |
|---|---|---|---|---|
| Ingredients | capsules | capsules | capsules | capsules |
| (wt %) | A | B | C | D |
| Whey protein | 1% | 1% | 1% | 1% |
| isolate (WPI) ¹⁾ | | | | |
| Chitosan | 0.5% | 0 | 0.5% | 0.5% |
| oligosaccharide | | | | |
| (Chi. Olig.) ²⁾ | | | | |
| DI water for | 44% | 34.3% | 34% | 48.5% |
| emulsifier | | | | |
| CaCl₂ • 2H₂O | 0 | 0.2% | 0.2% | 0 |
| DI water for | 0 | 10% | 9.8% | 0 |
| CaCl₂ • 2H₂O | | | | |
| dissolution | | | | |
| Perfume oil ³⁾ | 20% | 20% | 20% | 20% |
| TEOS ⁴⁾ | 10% | 10% | 10% | 10% |
| 1 mM HCl | 20% | 20% | 20% | 20% |
| solution ⁵⁾ | | | | |
| (HCl diluted in | | | | |
| DI water) | | | | |
| APTES ⁶⁾ | 4.5% | 4.5% | 4.5% | 0 |

¹⁾ Whey protein isolate (Bipro)
²⁾ β-1,4-oligo-glucosamine, M.W. <3000 (Aoxing Biotechnology, Zhejiang, China), M.W. 1500
³⁾ Perfume oil A (see Table 2)
⁴⁾ Tetraethyl orthosilicate
⁵⁾ Hydrochloric acid
⁶⁾ (3-Aminopropyl)triethoxysilane

TABLE 2
Perfume oil A composition

| Ingredient | Parts |
|---|---|
| Cyclosal¹⁾ | 20 |
| Verdox ²⁾ | 20 |
| Dorisyl ³⁾ | 20 |
| Romascone ⁴⁾ | 20 |
| Salicynile ⁵⁾ | 20 |

¹⁾3-(4-isopropylphenyl)-2-methylpropanal
²⁾ Tert-Butyl-1-Cyclohexyl Acetate
³⁾ 4-(Tert-Butyl)cyclohexyl acetate
⁴⁾ (Methyl 2,2-dimethyl-6-methylene-1-cyclohexanecarboxylate
⁵⁾ (2Z)-2-phenyl-2-hexenenitrile

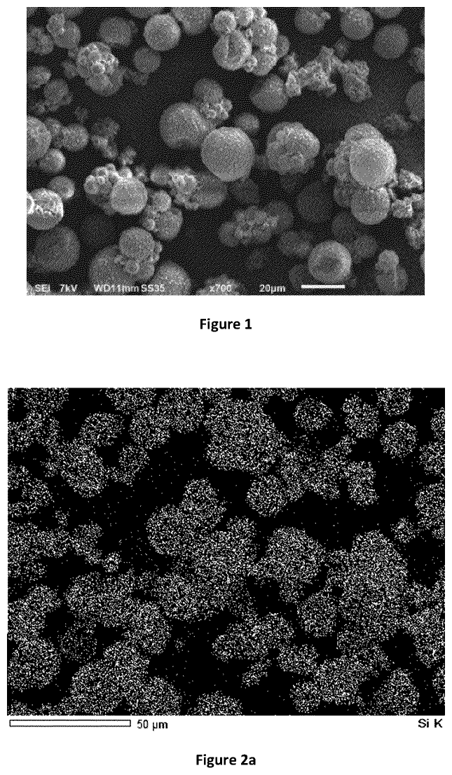

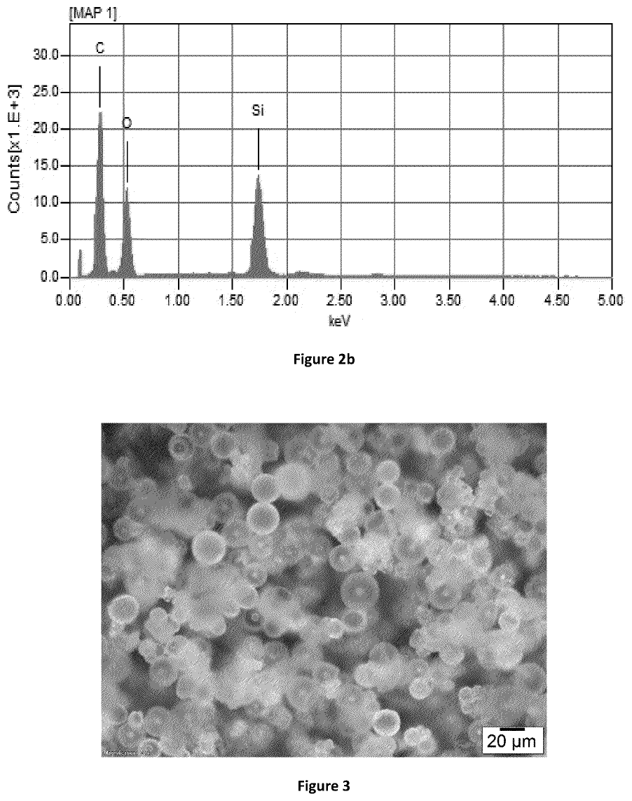

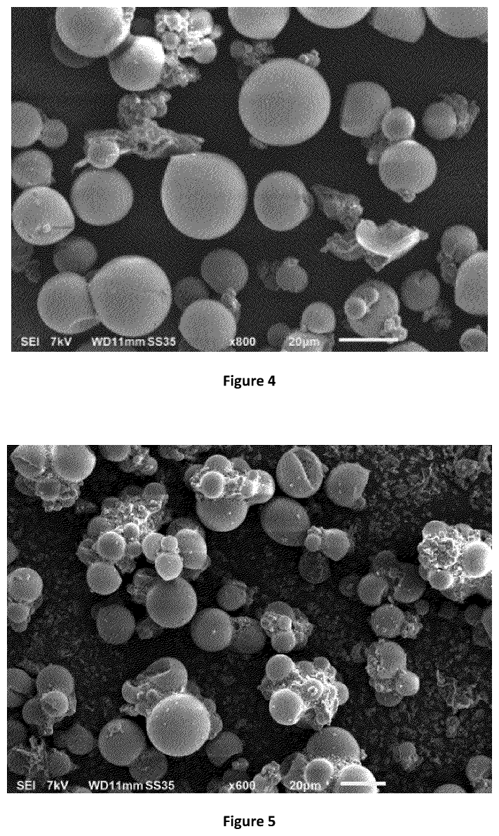

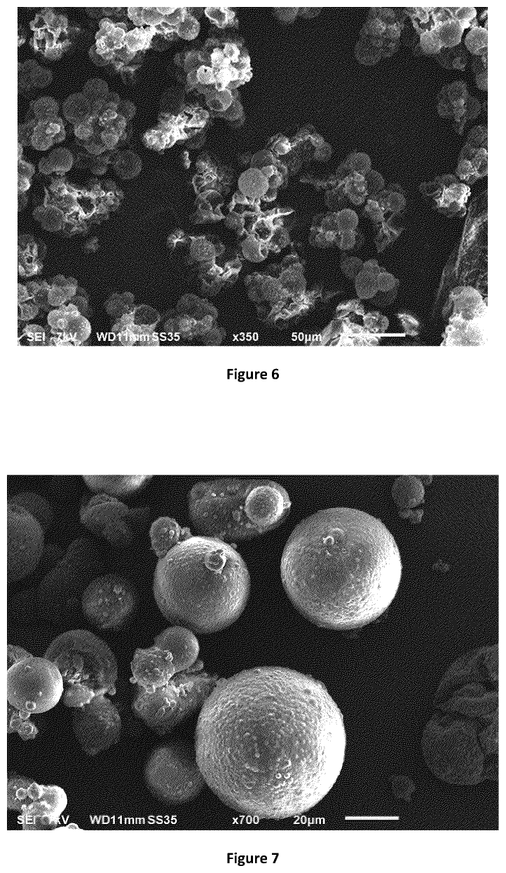

As can be seen from FIG. 1 to FIG. 6, microcapsules were obtained. FIG. 2 presents SEM-EDS silicon (Si) elemental mapping and EDS spectra of FIG. 1 region, which shows microcapsules shell is (composed of) silicon-based material. Microcapsules are stable upon drying on the glass slide and SEM imaging.

### Example 2

### Preparation of Microcapsules by the Process of the Invention

Microcapsules E-G were prepared using the same protocol as described in Example 1 except that for:
Sample E: TEOS was directly added to the emulsion
Sample F: TEOS was directly added in the oil phase
Sample G: TEOS was added in the oil phase (5%) and in the emulsion (10%, perhydrolysis with 1 mM HCl)

TABLE 3
Microcapsules composition

| Ingredients | | Microcapsules | |
|---|---|---|---|
| (wt %) | Microcapsules E | F | Microcapsules G |
| Whey protein | 1% | 1% | 1% |
| isolate (WPI) ¹⁾ | | | |
| Chitosan | 0.5% | 0.5% | 0.5% |
| oligosaccharide | | | |
| (Chi. Olig.) ²⁾ | | | |
| DI water for | 64% | 64% | 39% |
| emulsifier | | | |
| Perfume oil ³⁾ | 20% | 20% | 20% |
| TEOS ⁴⁾ | 10% | 10% | 15% |
| 1 mM HCl | 0% | 0% | 20% |
| solution ⁵⁾ | | | |
| (HCl diluted in | | | |
| DI water) | | | |
| APTES ⁶⁾ | 4.5% | 4.5% | 4.5% |

¹⁾ Whey protein isolate (Bipro)
²⁾ β-1,4-oligo-glucosamine, M.W. <3000 (Aoxing Biotechnology, Zhejiang, China), M.W. 1500
³⁾ Perfume oil A (see Table 2)
⁴⁾ Tetraethyl orthosilicate
⁵⁾ Hydrochloric acid
⁶⁾ (3-Aminopropyl)triethoxysilane

### Example 3

### Preparation of Microcapsules by the Process of the Invention

Microcapsules H and I have been prepared according the following protocol.

The reaction steps:
1) Dissolve shellac in ethanol, stirring at 50° C. At the right concentration, shellac can fully dissolve in ethanol, which stays as clear solution at room temperature.
2) At room temperature, pipette shellac/ethanol solution into perfume oil under stirring. Continue stirring and heat up the mixture to 80° C. to achieve a homogenous solution, cool down to room temperature.
3) Whey protein isolate and chitosan oligosaccharide are added to DI water at RT and stirred for ˜30 mins at RT, until no dry powder or dry materials are observed in the solution.
4) The shellac/ethanol/perfume oil is combined with WPI/Chitosan Oligosaccharide solution, and homogenized (10,000 rpm for 2 mins) to create oil-in-water emulsion.
5) The emulsion is transferred to a reactor and stirred at RT;
6) The reactor is heated to 80° C. and held for 2 hours before cooling to RT;
7) Meanwhile, TEOS is added to 1 mM HCl solution in a vial and sonicated for 20 mins;
8) The pre-hydrolyzed TEOS solution is added to the reactor; and the emulsion is stirred at RT for 2.5 hrs.
9) Optionally, APTES is added to the reactor, and the emulsion is stirred at RT for 20 hrs.

TABLE 4
Microcapsules composition

| Ingredients (wt %) | Microcapsules H | Microcapsules I |
|---|---|---|
| Shellac | 2% | 2% |
| (Poly-Soleil SY 400) | | |
| Ethanol, 200 proof | 8% | 8% |
| Perfume oil A | 20% | 20% |
| Whey protein isolate | 1% | 1% |
| Chitosan | 0.5% | 0.5% |
| Oligosaccharide | | |
| DI water for emulsifier | 38.50% | 34% |
| TEOS | 10% | 10% |
| 1 mM HCl solution | 20% | 20% |
| APTES | 0% | 4.5% |

Microcapsules H are shown in FIG. 7.

### Example 4

### Preparation of Spray-Dried Microcapsules

Microcapsules J were prepared using the same protocol as described in Example 1 except for: Uvinul A Plus is added and dissolved in perfume oil prior to emulsification. Chitosan Oligosaccharide with M. W. of 1052 is used.

TABLE 5
Microcapsules composition

| Ingredients | Microcapsules |
|---|---|
| (wt %) | J |
| Whey protein | 1% |
| isolate (WPI) ¹⁾ | |
| Chitosan | 0.5% |
| oligosaccharide | |
| (Chi. Olig.) ²⁾ | |
| DI water for | 44% |
| emulsifier | |
| Perfume oil A³⁾ | 19% |
| Uvinul A Plus⁴⁾ | 1% |
| TEOS⁵⁾ | 10% |
| 1 mM HCl | 20% |
| solution ⁶ | |
| (HCI diluted in | |
| DI water) | |
| APTES ⁷⁾ | 4.5% |

1) Whey protein isolate (Bipro)
2) β-1,4-oligo-glucosamine, M. W.<3000 (Aoxing Biotechnology, Zhejiang, China), M. W. 1052
3) Perfume oil A (see Table 2)
4) UV Tracer
5) Tetraethyl orthosilicate
6) Hydrochloric acid
7) (3-Aminopropyl)triethoxysilane

Microcapsules J are first washed and rinsed with DI water.

Spray drying procedure is the following:
1) Dissolve Capsul® (modified starch) and Maltodextrin 10DE into water at 600 rpm, 50° C. for 2 hours
2) Add capsule slurry to carrier solution and gently stir
3) Turn on spray dryer and allow to reach operating temperature
4) Run 50 mL hot DI water through spray dryer
5) Run capsule and carrier mixture through spray dryer while stirring mixture
6) When finished spray drying, clean lines with hot DI water
7) Cool down spray dryer and remove samples from coarse collector and fines collector

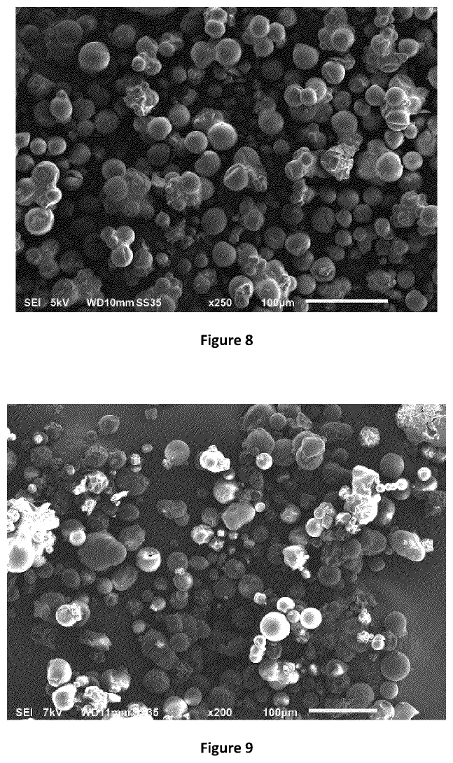

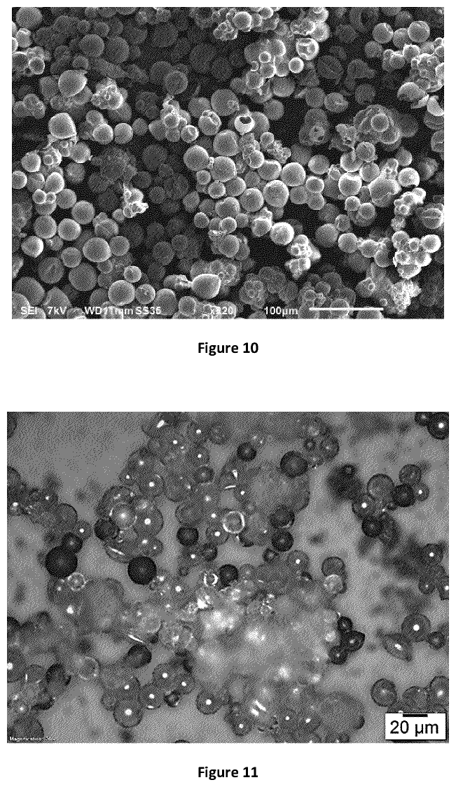

FIGS. 8-10 show SEM electron microscopy images of microcapsules J after rinsing, spray drying (coarse collector fraction) and after rehydration to dissolve the carrier revealing microcapsules are intact following the spray drying and resuspension processes.

### Example 5

### Preparation of Microcapsules by the Process of the Invention

### Long Chain Silanes in the Oil Phase

Microcapsules K-L were prepared according to the following protocol and using the following components.

TABLE 6
Microcapsules composition

| | Microcapsules | Microcapsules |
|---|---|---|
| Ingredients (wt %) | K | L |
| Whey protein isolate | 1% | 1% |
| (WPI) ¹⁾ | | |
| Chitosan | 0.5% | 0.5 |
| oligosaccharide (Chi. | | |
| Olig.) ²⁾ | | |
| DI water for emulsifier | 44% | 44% |
| Perfume oil ³⁾ | 20% | 20% |
| Triethoxy-n-octylsilane | 3% | 0 |
| Dodecyltriethoxysilane | 0 | 3% |
| TEOS ⁴⁾ | 7% | 7% |
| 1 mM HCl solution ⁵⁾ | 2 0% | 20% |
| (HCI diluted in DI | | |
| water) | | |
| APTES ⁶⁾ | 4.5% | 4.5% |

¹⁾ Whey protein isolate (Bipro)
²⁾ β-1,4-oligo-glucosamine, M.W. <3000 (Aoxing Biotechnology, Zhejiang, China), M.W. 1480
³⁾ Perfume oil A (see Table 2)
⁴⁾ Tetraethyl orthosilicate
⁵⁾ Hydrochloric acid
⁶⁾ (3-Aminopropyl)triethoxysilane

1) Whey protein isolate are added to DI water at RT and stirred to dissolve, then chitosan oligosaccharide is added, and stirred for about 30 mins at RT until no dry powder or dry materials are observed in the solution. The pH of the mixture solution is adjusted to 5.5 using 0.1 M HCl.
2) Long chain silane (Triethoxy-n-octylsilane or Dodecyltriethoxysilane) is added to perfume oil and stirred to achieve a homogenous mixture.
3) The perfume oil with long chain silane is combined with WPI/Chitosan Oligosaccharide solution, and homogenized (10,000 rpm for 2 mins) to create oil-in-water emulsion.
4) The emulsion is transferred to a reactor and stirred at RT;
5) Meanwhile, TEOS is added to 1 mM HCl solution in a separate vial and sonicated for 20 mins;
6) The pre-hydrolyzed TEOS solution is added to the reactor; and the emulsion is stirred at RT for 30 mins;
7) Then the reactor is heated up to 80° C. and held at 80° C. for 2 hours before cooling to RT;
8) APTES is added to the reactor, and the emulsion is stirred at RT for 20 hrs.

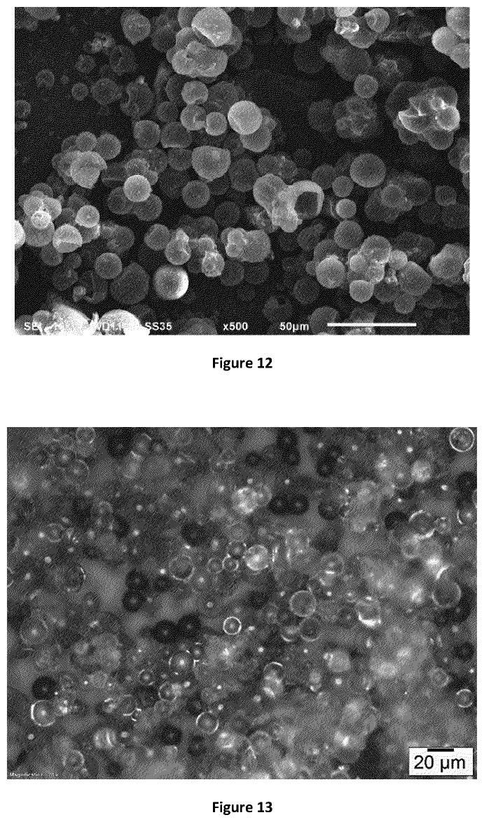

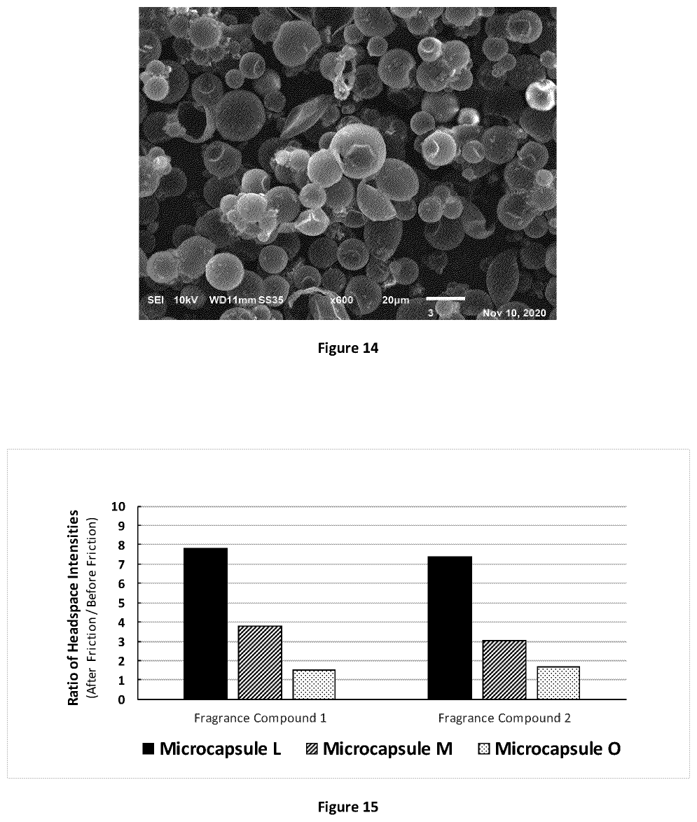

FIG. 11-14 show optical image of microcapsules K and L dried on glass slide and SEM electron images of microcapsules K and L. (Microcapsules are stable upon drying on the glass slide and SEM imaging.)

### Example 6

### Preparation of Microcapsules by the Process of the Invention

Microcapsules M-O were prepared using the same protocol as described in Example 1 except that for
1) WPI is first dissolved in DI, then chitosan oligosaccharide is added to the solution, stir for 30 min until no dry powder in the solution. The pH of the mixture solution is adjusted to 5.5 using diluted HCl (0.1 M HCl solution). (Chitosan Oligosaccharide with M. W. of 1480 is used)

TABLE 7
Microcapsules composition

| | Microcapsules | Microcapsules | Microcapsules |
|---|---|---|---|
| Ingredients (wt %) | M | N | O |
| Whey protein isolate | 1% | 1% | 1% |
| (WPI) ¹⁾ | | | |
| Chitosan | 0.5% | 0.5% | 0.5% |
| oligosaccharide (Chi. | | | |
| Olig.) ²⁾ | | | |
| DI water for emulsifier | 44% | 44% | 44% |
| Perfume oil ³⁾ | 20% | 20% | 20% |
| TEOS ⁴⁾ | 10% | 10% | 10% |
| 1 mM HCl solution ⁵⁾ | 20% | 20% | 0% |
| (HCl diluted in DI | | | |
| water) | | | |
| 2.5 mM HCl solution ⁵⁾ | 0% | 0% | 20% |
| (HCl diluted in DI | | | |
| water) | | | |
| APTES ⁶⁾ | 4.5% | 0% | 4.5% |

¹⁾ Whey protein isolate (Bipro)
²⁾ β-1,4-oligo-glucosamine, M.W. <3000 (Aoxing Biotechnology, Zhejiang, China), M.W. 1480
³⁾ Perfume oil A (see Table 2)
⁴⁾ Tetraethyl orthosilicate
⁵⁾ Hydrochloric acid
⁶⁾ (3-Aminopropyl)triethoxysilane

### Example 7

### Using Dynamic Headspace to Evaluate Microcapsule Encapsulation and Pop Effect

To assess the pop effect of capsules after rubbing, dynamic headspace intensity measurements were performed on blotter papers which were loaded with 1% microcapsules slurry and evaluated after drying, before and after applying friction by rubbing with a gloved finger. 100 uL of 1% capsules slurry (0.2% perfume oil content of a 5 compound perfume mixture [equal mass of each of 5 compounds as described in Table 2) of Microcapsules L, M and O were pipetted onto 0.5 inch×1.5 inch rectangular blotter paper strips and dried for 24 hrs under ambient conditions. The dosed and dried blotter strips were then carefully placed in 20 mL glass dynamic headspace vials and then the vials capped. For the ‘after rubbing’ samples, the blotter strips were rubbed with 3 vigorous passes using a gloved index finger. The rubbed blotters were then placed in the vials and capped. The headspace measurements of fragrance intensity of the blotters before and after friction were conducted using a Shimadzu GC-MS instrument with DHS (dynamic headspace) capabilities. The sampling phase was followed by a trapping phase of 20 ml of HS onto a Tenax tube, then desorption for GC-MS analysis and interpretation.

The ratios of peak headspace signals before and after rubbing for all five perfumery ingredients were determined. The ratios of the selected characteristic headspace signals for Dorisyl (fragrance compound 1) and Verdox (fragrance compound 2) were chosen to illustrate the rubbing effect as shown in FIG. 15. The ratio values (ratio=headspace intensity value after friction/headspace intensity value before friction) are significantly higher than 1 for Microcapsules L, M and O, indicating a stronger signal after rubbing and significant pop effect upon applying friction to release the fragrance oil from the capsule core. The rubbing burst effect was especially pronounced for Microcapsules L and M, with headspace intensity ratios greater than 2.

FIG. 15 demonstrates the plot of ratios of headspace intensities for Microcapsules L, M and O dosed onto paper blotters and evaluated before and after applying friction to illustrate pop effect.

### Example 8

### Zeta Potential Measurements

Zeta potential of microcapsules according to the invention (M, N, L and O) were measured in 1 mM KCl at pH 3, 5.5, and 9 using a Malvern ZetaSizer Nano ZS-90. The results show that microcapsules M, N and O prepared with APTES have positively charged zeta potentials greater than +40 mV at pH 5.5.

TABLE 9
Zeta potentials of microcapsules M, N, L, O

| Zeta | Microcapsules | Microcapsules | Micro- | Microcapsules |
|---|---|---|---|---|
| potential | M | N (no APTES) | capsules L | O |
| At pH 9 | 22.9 ± 1.19 | −21.2 ± 1.18 | NE | NE |
| At pH 5.5 | 42.9 ± 1.70 | 5.89 ± 0.46 | 46.3 ± 0.26 | 43.0 ± 1.40 |
| At pH 3 | 63.0 ± 2.26 | 24.9 ± 2.22 | NE | NE |

NE: not evaluated

### Example 9

### Microcapsules a after Incubation in Fabric Softener

Microcapsules A was incubated in fabric softener (see composition in Table 10) for 2 months at 37° C. in a closed jar.

TABLE 10
Fabric Conditioner composition

| Product | Wt % |
|---|---|
| Stepantex VL 90A | 8.88 |
| Calcium Chloride Sol. 10% | 0.36 |
| Proxel GXL | 0.04 |
| Perfume | 1 |
| Water | 89.72 |
| TOTAL | 100 |

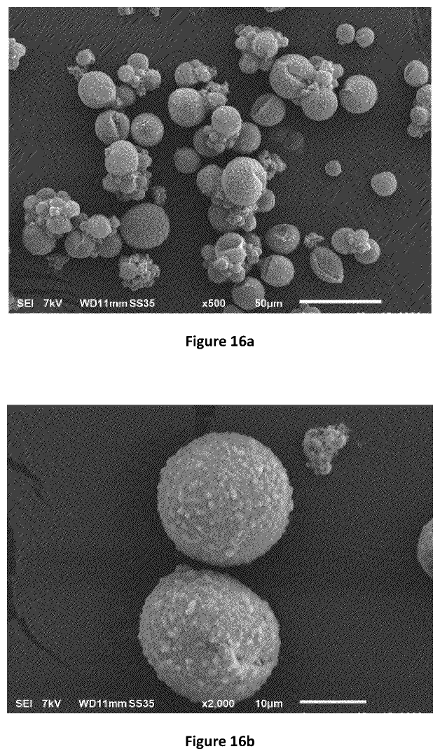

FIG. 16 is SEM electron micrographs of Microcapsules A after incubation, showing microcapsules A can retain capsule structure in the fabric softener during the incubation period.

### Example 10

Microcapsules A underwent an extreme heat treatment to illustrate that shells can maintain their structure, after prolonged heat exposure (500° C.). These microcapsules were further assessed by elemental analysis to show presence of elemental silicon after heating.

The heat exposure test is performed using TA instruments TGA Q50. Microcapsules A slurry was loaded on the TGA sample pan, and heated to 500° C. following this protocol: after initial equilibration at 30° C., ramp at 5° C./min to 50° C., isothermal at 50° C. for 250 min, ramp at 10° C./min to 500° C. and hold at 500° C. isothermal for 60 min.

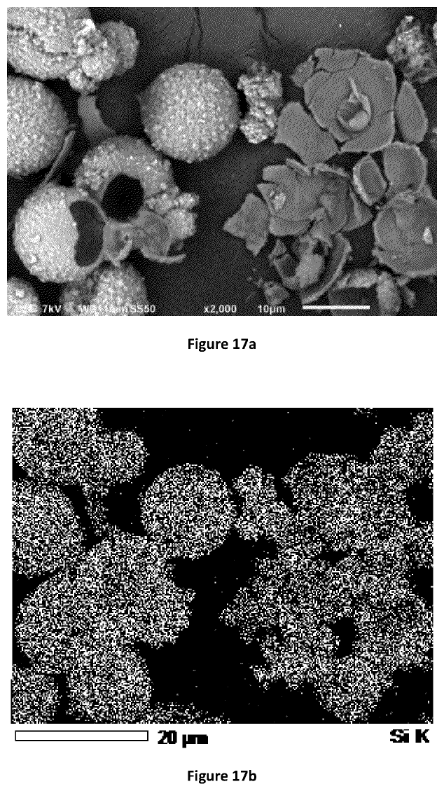

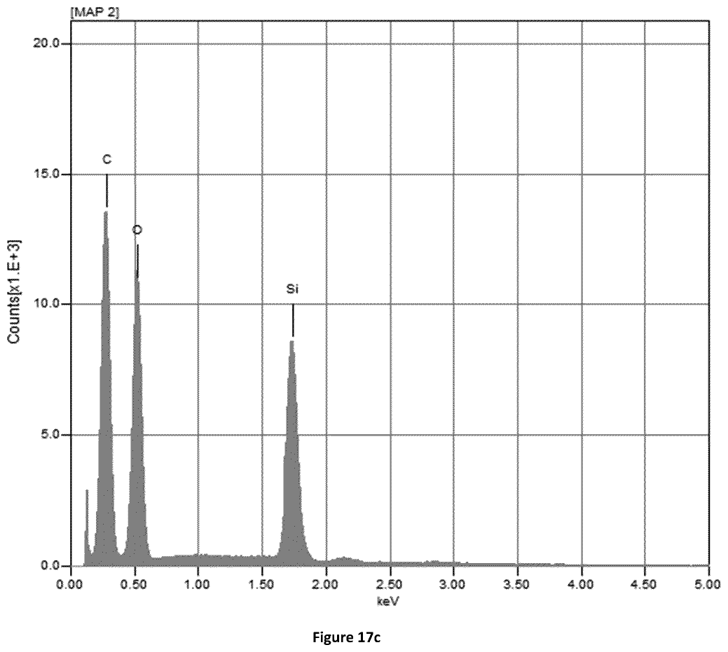

FIG. 17 shows Back Scattering electron microscopy image, silicon (Si) elemental EDS mapping and SEM-EDS (energy dispersive spectroscopy) spectra of microcapsules A after exposure to 500° C. illustrating that microcapsules can maintain their physical structure following extreme heat treatments.

### Example 11

### Liquid Detergent Composition

Microcapsule slurry (see examples 1-6) is dispersed in a liquid detergent composition to obtain a concentration of encapsulated perfume of 0.15%.

TABLE 11
Liquid detergent composition

| | Concentration |
|---|---|
| Ingredients | [wt %] |
| Sodium C₁₄₋₁₇ Alkyl Sec Sulfonate¹⁾ | 7 |
| Fatty acids, C₁₂₋₁₈ and C₁₈-unsaturated²⁾ | 7.5 |
| C_{12/14} fatty alcohol polyglycol | 17 |
| ether with 7 mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 Methacrylate | 6 |
| structuring Crosspolymer⁵⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Origin: Genencor International
⁵⁾Aculyn 88; Origin: Dow Chemical

### Example 12

### Rinse-Off Conditioner

Microcapsule slurry (see examples 1-6) is dispersed in a rinse-off conditioner base described in table 10 to obtain a concentration of encapsulated perfume oil at 0.5%.

TABLE 12
Rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | TOTAL: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

### Example 13

### Shampoo Composition

Microcapsule slurry (see examples 1-6) is dispersed in a shampoo composition to add the equivalent of 0.2% perfume.

TABLE 13
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous | q.s. |
| | sol. till pH 5.5-6 | |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 14

### Antiperspirant Roll-on Emulsion Composition

Microcapsule slurry (see examples 1-6) is dispersed in antiperspirant roll-on emulsion composition to add the equivalent of 0.2% perfume.

TABLE 14
Antiperspirant composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin : ICI
²⁾BRIJ 721; origin : ICI
³⁾ARLAMOL E; origin : UNIQEMA-CRODA
⁴⁾LOCRON L; origin : CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 15

### Shower-Gel Composition

Microcapsule slurry (see examples 1-6) is dispersed in the following composition to add the equivalent of 0.2% perfume.

TABLE 15
Shower gel composition

| Ingredients | Amount (% wt) | Function |
|---|---|---|
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% | 1.000 | pH adjuster |
| aqueous solution | | |
| Cocamidopropyl Betaine⁴⁾ | 8.000 | Surfactant |
| Methylchloroisothiazolinone | 0.100 | Preservative |
| and Methylisothiazolinone⁵⁾ | | |
| Citric Acid (40%) | 0.500 | pH adjuster |

⁷⁾ EDETA B POWDER; trademark and origin: BASF
⁸⁾ CARBOPOL AQUA SF-1 POLYMER; trademark and origin: NOVEON
⁹⁾ ZETESOL AO 328 U; trademark and origin: ZSCHIMMER & SCHWARZ
¹⁰⁾ TEGO-BETAIN F 50; trademark and origin: GOLDSCHMIDT
¹¹⁾ KATHON CG; trademark and origin: ROHM & HASS

1. A process for preparing a core-shell microcapsule slurry, said process comprising the steps of:
(i) Mixing in a dispersing phase a protein and a polycation;
(ii) Adding an oil phase comprising a hydrophobic material to the dispersing phase to form a dispersion;
(iii) performing a curing step to form a microcapsule slurry;
wherein at least one silicon precursor is added in step (i) and/or in step (ii) and/or in step (iii).

2. The process according to claim 1, wherein the protein is chosen from the group consisting of potato protein, chickpea protein, algae protein, faba bean protein, barley protein, oat protein, wheat gluten protein, lupin protein, whey protein, milk proteins, caseinate salts, sodium caseinate or calcium caseinate, casein, hydrolyzed proteins, gelatins, gluten, pea protein, soy protein, silk protein, beta-lactoglobulin, ovalbumine, bovine serum albumin, and mixtures thereof.

3. The process according to claim 1, wherein the polycation is chosen from the group consisting of chitosan, chitosan oligomer, chitosan oligosaccharide, Ca²⁺, Mg²⁺, Zn²⁺, Ba²⁺, Sr²⁺, and mixtures thereof.

4. The process according to claim 3, wherein the polycation is a chitosan oligosaccharide having a molecular weight lower than 5000.

5. The process according to claim 1, wherein the weight ratio between the protein and the polycation is comprised between 5:1 and 1:3.

6. The process according to claim 1, wherein the protein is whey protein and wherein the polycation is chitosan oligosaccharide.

7. The process according to claim 1, wherein the at least one silicon precursor is chosen from the group consisting of tetraethyl orthosilicate (TEOS), tetramethyl orthosilicate (TMOS), triethoxymethylsilane, dimethyldimethoxysilane, ethyltriethoxysilane, amine functionalized silanes, (3-aminopropyl)triethoxysilane, (3-aminopropyl)trimethoxysilane, N-(2-aminoethyl)-3-aminopropyltriethoxysilane, N-dimethyl-3-aminopropylmethyldimethoxysilane, 3-aminopropylmethyldiethoxysilane, 4-aminobutylthiethoxysilane, and mixtures thereof.

8. The process according to claim 1, wherein the at least one silicon precursor is added in the dispersion obtained in step ii).

9. The process according to claim 1, wherein at least a first silicon precursor is added in the dispersion obtained in step ii) and wherein at least a second silicon precursor is added during or after step (iii).

10. The process according to claim 9, wherein the first silicon precursor is tetraethyl orthosilicate and wherein the second silicon precursor is (3-aminopropyl)triethoxysilane.

11. The process according to claim 1, wherein long and/or medium chain silane or mixture of silanes are added to the oil phase.

12. The process according to claim 1, wherein no polyfunctional monomer is added at any stage of the process.

13. A core-shell microcapsule comprising:
an oil-based core comprising a hydrophobic material, and
a composite shell comprising
(i) a biopolymer-based material comprising complexes made of a protein and a polycation, and
(ii) a silicon-based material.

14. The core-shell microcapsule according to claim 13, wherein the protein is whey protein and wherein the polycation is chitosan oligosaccharide.

15. A consumer product in the form of a perfumed consumer product or a flavoured consumer product comprising the microcapsules as defined in claim 13.

16. The process according to claim 1, wherein the hydrophobic material is a perfume or a flavor.

17. The process according to claim 4, wherein the polycation is a chitosan oligosaccharide having a molecular weight lower than 3000.

18. The process according to claim 5, wherein the weight ratio between the protein and the polycation is comprised between 3:1 and 1:2.

19. The process according to claim 6, wherein the protein is whey protein isolate.

20. The process according to claim 11, wherein the long and/or medium chain silane or mixture of silanes are chosen from silanes with organic chain substitution of more than 3 carbons, triethoxy-n-octylsilane, dodecyltriethoxysilane, octadecyltriethoxysilane, decyltriethoxysilane, n-hexyltriethoxysilane and hexadecyltriethoxysilane, and mixtures thereof.
